(19) United States
(12) Patent Application Publication
(10) Pub. No.: US 2023/0082968 A1
(43) Pub. Date: Mar. 16, 2023

(54) EXOGENOUS NITRIC OXIDE FOR IMPROVED SUSCEPTIBILITY AND LOWERED ANTIBIOTIC RESISTANCE IN RESISTANT RESPIRATORY BACTERIA

(71) Applicant: The University of North Carolina at Chapel Hill, Chapel Hill, NC (US)

(72) Inventors: MARK H. SCHOENFISCH, Chapel Hill, NC (US); KAITLYN ROSE ROUILLARD, Durham, NC (US)

(21) Appl. No.: 17/799,871

(22) PCT Filed: Feb. 11, 2021
(86) PCT No.: PCT/US2021/017711

§ 371 Date: Aug. 15, 2022

Related U.S. Application Data
(60) Provisional application No. 62/976,738, filed on Feb. 14, 2020.
(60) Provisional application No. 63/125,865, filed on Dec. 15, 2020.

(51) Int. Cl. A61K 47/61; A61K 31/427; A61K 31/546; A61K 31/496; A61K 38/12; A61K 31/407; A61K 31/7036; A61P 31/04

(52) U.S. Cl. CPC A61K 47/61; A61K 31/407; A61K 31/427; A61K 31/496; A61K 31/546; A61K 31/7036; A61K 38/12; A61P 31/04

(57) ABSTRACT

Disclosed are antimicrobial compositions and formulations directed against a broad range of bacteria and Pseudomonas aeruginosa biofilms. In an aspect, the compositions and formulations do not engender resistance. In an aspect, in combination with antibiotics, the compositions and formulations slow the development of antibiotic-resistance and greatly improve bacterial susceptibility to multiple classes of antibiotics.

### CROSS REFERENCE TO RELATED APPLICATIONS

This application claims priority from and the benefit of U.S. Provisional Pat. Application Nos. 62/976,738 and 63/125,865, filed Feb. 14, 2020 and Dec. 15, 2020, respectively, which are hereby incorporated by reference in their entireties for all purposes.

### FIELD

The present disclosure relates to antimicrobial compositions and formulations directed against a broad range of bacteria and Pseudomonas aeruginosa biofilms, without engendering resistance. The present disclosure further relates to antimicrobial compositions and formulations which, in combination with antibiotics, slow the development of antibiotic-resistance and greatly improve bacterial susceptibility to multiple classes of antibiotics.

The ESKAPE pathogens ( Enterococcus faecium, Staphylococcus aureus, Klebsiella pneumoniae, Acinetobacter baumannii, Pseudomonas aeruginosa, and Enterobacter spp.) are able to “escape” antibiotic action, frequently multidrug-resistant (MDR) and the leading cause of nosocomial infections worldwide [Santajit, S. & Indrawattana, N. Mechanisms of Antimicrobial Resistance in ESKAPE Pathogens. Biomed Res. Int. 2016, 1-8 (2016); Ciofu, O. & Tolker-Nielsen, T. Tolerance and Resistance of Pseudomonas aeruginosa Biofilms to Antimicrobial Agents-How P. aeruginosa Can Escape Antibiotics. Front. Microbiol. 10, 913 (2019); Mulani, M. S., Kamble, E. E., Kumkar, S. N., Tawre, M. S. & Pardesi, K. R. Emerging Strategies to Combat ESKAPE Pathogens in the Era of Antimicrobial Resistance: A Review. Front. Microbiol. 10, (2019)]. Respiratory tract infections frequently include ESKAPE pathogens like P. aeruginosa and S. aureus, which are hypermutable and intrinsically resistant [Ciofu, O. & Tolker-Nielsen, T. Tolerance and Resistance of Pseudomonas aeruginosa Biofilms to Antimicrobial Agents-How P. aeruginosa Can Escape Antibiotics. Front. Microbiol. 10, 913 (2019); Davies, E. V., James, C. E., Brockhurst, M. A. & Winstanley, C. Evolutionary diversification of Pseudomonas aeruginosa in an artificial sputum model. BMC Microbiol. 17, 3 (2017); Fernandez-Barat, L. et al. Phenotypic shift in Pseudomonas aeruginosa populations from cystic fibrosis lungs after 2-week antipseudomonal treatment. J. Cyst. Fibros. 16, 222-229 (2017)]. Additionally, these pathogens form biofilms which are protected by airway mucus, where antibiotics lose their efficacy due in part to poor diffusion and low bioactivity [Davies, E. V., James, C. E., Brockhurst, M. A. & Winstanley, C. Evolutionary diversification of Pseudomonas aeruginosa in an artificial sputum model. BMC Microbiol. 17, 3 (2017); Caraher, E. M. et al. The effect of recombinant human lactoferrin on growth and the antibiotic susceptibility of the cystic fibrosis pathogen Burkholderia cepacia complex when cultured planktonically or as biofilms. J. Antimicrob. Chemother. 60, 546-554 (2007); Cullen, L. & McClean, S. Bacterial adaptation during chronic respiratory infections. Pathogens 4, 66-89 (2015); Müller, L. et al. Human airway mucus alters susceptibility of Pseudomonas aeruginosa biofilms to tobramycin, but not colistin. J. Antimicrob. Chemother. 73, 2762-2769 (2018); Mendelman, P. M. et al. Aminoglycoside penetration, inactivation, and efficacy in cystic fibrosis sputum. Am. Rev. Respir. Dis. 132, 761-765 (1985)]. Nitric oxide (NO), an endogenously produced free radical species involved in the immune response, has previously been shown to reduce airway mucus viscosity and also exert broad-spectrum antibacterial and antibiofilm action against several ESKAPE pathogens [Reighard, K. P., Hill, D. B., Dixon, G. A., Worley, B. V. & Schoenfisch, M. H. Disruption and eradication of P. aeruginosa biofilms using nitric oxide-releasing chitosan oligosaccharides. Biofouling 31, 775-87 (2015); Ahonen, M. J. R., Hill, D. B. & Schoenfisch, M. H. Nitric oxide-releasing alginates as mucolytic agents. ACS Biomater. Sci. Eng. 5, 3409-3418 (2019); Schairer, D. O., Chouake, J. S., Nosanchuk, J. D. & Friedman, A. J. The potential of nitric oxide releasing therapies as antimicrobial agents. Virulence 3, 271-279 (2012); Hetrick, E. M. et al. Bactericidal efficacy of nitric oxide-releasing silica nanoparticles. ACS Nano 2, 235-246 (2008)].

The solution to this technical problem is described herein.

### BRIEF SUMMARY

Antibiotic resistance in bacteria is a major global threat and the leading cause for healthcare-related morbidity and mortality [Friedman, N. D., Temkin, E. & Carmeli, Y. The negative impact of antibiotic resistance. Clin. Microbiol. Infect. 22, 416-422 (2016); Ragheb, M. N. et al. Inhibiting the Evolution of Antibiotic Resistance. Mol. Cell 73, 157-165.e5 (2019)]. According to the U.S. Centers for Disease Control, resistant infections are expected to kill 10 million people by 2050 [Centers for Disease Control. Antibiotic Resistance Threats in the United States, 2013. Antibiotic Resistant Threats in the United States (2013)]. Resistant respiratory infections are particularly difficult to treat because they form protective biofilms inside airway mucus and can survive for decades [Kovach, K. et al. Evolutionary adaptations of biofilms infecting cystic fibrosis lungs promote mechanical toughness by adjusting polysaccharide production, npj Biofilms Microbiomes 3, (2017)]. Novel antimicrobial agents that are capable of fully eradicating resistant infections, even those deep inside the lower airway, are desperately needed along with strategies for combatting the rise of antibiotic resistant bacteria. Here, the applicants show that nitric oxide behaves as a broad-spectrum antimicrobial and improves antibiotic efficacy. Alone, it kills a broad range of bacteria and Pseudomonas aeruginosa biofilms without engendering resistance. In combination with antibiotics, it slows the development of antibiotic-resistance and greatly improves bacterial susceptibility to multiple classes of antibiotics. No antimicrobial on the market today combines broad-spectrum antimicrobial efficacy, biofilm killing, synergism with conventional antibiotics, and increase of antibiotic susceptibility in an “all-in-one” therapy. The use of nitric oxide in combination with antibiotics as described herein can eradicate a multitude of antibiotic-resistant infections, even those protected by a diseased state airway.

The following embodiments are contemplated.

Embodiment 1. A method of increasing susceptibility of a microorganism to at least one antibiotic, comprising: contacting the microorganism with a nitric oxide-releasing chitosan oligosaccharide (COS/NO) for a period, and contacting the microorganism with the at least one antibiotic. Said contacting may be concurrent, sequential, or a combination thereof.

Embodiment 2. The method of embodiment 1, wherein the microorganism is selected from the genera consisting of: Enterococcus, Staphylococcus, Klebsiella, Acinetobacter, Pseudomonas, and Enterobacter.

Embodiment 3. The method of embodiment 1, wherein the microorganism is selected from the group consisting of: Enterococcus faecium, Staphylococcus aureus, Klebsiella pneumoniae, Acinetobacter baumannii, Pseudomonas aeruginosa, and Enterobacter spp.

Embodiment 4. The method of any one of embodiments 1-3, wherein the COS/NO: has a minimum inhibitory concentration (MIC) relative to the microorganism; and is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

Embodiment 5. The method of any one of embodiments 1-4, wherein the at least one antibiotic: has a minimum inhibitory concentration (MIC) relative to the microorganism; and is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

Embodiment 6. The method of any one of embodiments 1-5, wherein the period is selected from the group consisting of at least 0.25, at least 0.5, at least 0.75, at least 1, at least 1.25, at least 1.5, at least 1.75, at least 2, at least 2.25, at least 2.5, at least 2.75, at least 3, at least 3.25, at least 3.5, at least 3.75, at least 4, at least 4.25, at least 4.5, at least 4.75, at least 5, at least 5.25, at least 5.5, at least 5.75, at least 6, at least 6.25, at least 6.5, at least 6.75, at least 7, at least 7.25, at least 7.5, at least 7.75, and at least 8 hours.

Embodiment 7. The method of any one of embodiments 1-6, wherein the increased susceptibility is lower viability of the at least one microorganism, measured as colony-forming units (CFU) per unit volume, after said contacting with the COS/NO for a period and subsequent contacting with the at least one antibiotic, as compared to contacting with either the COS/NO alone or the at least one antibiotic alone.

Embodiment 8. The method of any one of embodiments 1-7, wherein the at least one microorganism is in a biofilm.

Embodiment 9. The method of any one of embodiments 1-8, wherein the COS/NO is in a composition formulated for: topical, oral, nasal, ophthalmic, intrathecal, parenteral, intraperitoneal, intravenous, subcutaneous, or intramuscular administration.

Embodiment 10. The method of any one of embodiments 1-9, wherein the at least one antibiotic is in a composition formulated for: topical, oral, nasal, ophthalmic, intrathecal, parenteral, intraperitoneal, intravenous, subcutaneous, or intramuscular administration.

Embodiment 11. The method of any one of embodiments 1-10, wherein the formulation is in the form of a paste, a liquid, a cream, a gel, a salve, a foam, an aerosol, a lotion, an ointment, a soap, a shampoo, a surgical drape, a suture, a bandage, a gauze, or a medical implant.

Embodiment 12. The method of any one of embodiments 1-11, wherein the at least one antibiotic is selected from the group consisting of: aminoglycosides, ansamycins, beta-lactams, carbacephems, carbapenems, cephalosporins, fluoroquinolones, glycopeptides, lincosamides, macrolides, monobactams, oxazolidinones, penicillins, phenicols, polypeptides, quinolones, streptogramins, sulfonamides, and tetracyclines.

Embodiment 13. The method of any one of embodiments 1-12, wherein the at least one antibiotic is selected from the group consisting of: aztreonam, ceftazidime, ciprofloxacin, colistin, meropenem, and tobramycin.

Embodiment 14. A method of reducing the development or progression, in a microorganism, of resistance to at least one antibiotic, comprising: contacting the microorganism with a nitric oxide-releasing chitosan oligosaccharide (COS/NO) for a period, and subsequently contacting the microorganism with the at least one antibiotic.

Embodiment 15. The method of embodiment 14, wherein the microorganism is selected from the genera consisting of: Enterococcus, Staphylococcus, Klebsiella, Acinetobacter, Pseudomonas, and Enterobacter.

Embodiment 16. The method of embodiment 14, wherein the microorganism is selected from the group consisting of: Enterococcus faecium, Staphylococcus aureus, Klebsiella pneumoniae, Acinetobacter baumannii, Pseudomonas aeruginosa, and Enterobacter spp.

Embodiment 17. The method of any one of embodiments 141-16, wherein the COS/NO: has a minimum inhibitory concentration (MIC) relative to the microorganism; and is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

Embodiment 18. The method of any one of embodiments 14-17, wherein the at least one antibiotic: has a minimum inhibitory concentration (MIC) relative to the microorganism; and is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

Embodiment 19. The method of any one of embodiments 14-18, wherein the period is selected from the group consisting of at least 0.25, at least 0.5, at least 0.75, at least 1, at least 1.25, at least 1.5, at least 1.75, at least 2, at least 2.25, at least 2.5, at least 2.75, at least 3, at least 3.25, at least 3.5, at least 3.75, at least 4, at least 4.25, at least 4.5, at least 4.75, at least 5, at least 5.25, at least 5.5, at least 5.75, at least 6, at least 6.25, at least 6.5, at least 6.75, at least 7, at least 7.25, at least 7.5, at least 7.75, and at least 8 hours.

Embodiment 20. The method of any one of embodiments 14-19, wherein the increased susceptibility is lower viability of the at least one microorganism, measured as colony-forming units (CFU) per unit volume, after said contacting with the COS/NO for a period and subsequent contacting with the at least one antibiotic, as compared to contacting with either the COS/NO alone or the at least one antibiotic alone.

Embodiment 21. The method of any one of embodiments 14-20, wherein the at least one microorganism is in a biofilm.

Embodiment 22. The method of any one of embodiments 14-21, wherein the COS/NO is in a composition formulated for: topical, oral, nasal, ophthalmic, intrathecal, parenteral, intraperitoneal, intravenous, subcutaneous, or intramuscular administration.

Embodiment 23. The method of any one of embodiments 14-22, wherein the at least one antibiotic is in a composition formulated for: topical, oral, nasal, ophthalmic, intrathecal, parenteral, intraperitoneal, intravenous, subcutaneous, or intramuscular administration.

Embodiment 24. The method of any one of embodiments 14-23, wherein the formulation is in the form of a paste, a liquid, a cream, a gel, a salve, a foam, an aerosol, a lotion, an ointment, a soap, a shampoo, a surgical drape, a suture, a bandage, a gauze, or a medical implant.

Embodiment 25. The method of any one of embodiments 14-24, wherein the at least one antibiotic is selected from the group consisting of: aminoglycosides, ansamycins, beta-lactams, carbacephems, carbapenems, cephalosporins, fluoroquinolones, glycopeptides, lincosamides, macrolides, monobactams, oxazolidinones, penicillins, phenicols, polypeptides, quinolones, streptogramins, sulfonamides, and tetracyclines.

Embodiment 26. The method of any one of embodiments 14-25, wherein the at least one antibiotic is selected from the group consisting of: aztreonam, ceftazidime, ciprofloxacin, colistin, meropenem, and tobramycin.

In one or more embodiments, the nitric oxide-releasing chitosan oligosaccharide (COS/NO) of Embodiment 1 is represented by the following structure disclosed in U.S. Pat. No. US 9850322 (incorporated by reference herein):
comprising at least one structural unit of formula I:

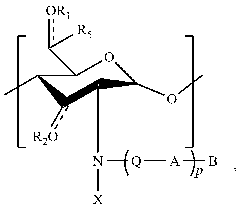

and optionally, at least one structural unit of formula II:

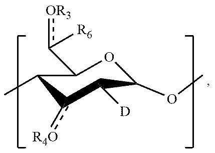

wherein, R₁, R₂, R₃ and R₄, if present, are each independently selected from the group consisting of hydrogen; C₁₋₅ alkyl(C═O)—, when the C₁₋₅ alkyl is methyl, Me(C═O)— is an acyl, Ac; and C₁₋₅ alkyl;

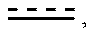

in each instance, is a single or double bond,
wherein in each instance where it is a double bond, R₁, R₂, R₃ or R₄ attached to the double bond-O is absent; when R₁ is absent, R₅ is hydrogen, hydroxyl, C₁₋₅ alkyl or C₁₋₅ alkoxy; when R₃ is absent, R₆ is hydrogen, hydroxyl, C₁₋₅ alkyl or C₁₋₅ alkoxy;
wherein in each instance where it is a single bond, R₁, R₂, R₃ or R₄ attached to the double bond-O is present; when R₁ is present, R₅ is hydrogen;
when R₃ is present, R₆ is hydrogen;
Q is —(CR_{c}R_{d})ᵥ—;
wherein R_{c} and R_{d} are independently hydrogen or C₁₋₅ alkyl, such as methyl, ethyl, n-propyl, isopropyl, t-butyl, n-butyl, isobutyl and pentyl. Preferably, R_{c} and R_{d} are independently hydrogen, methyl or ethyl; and v is an integer from 2 to 6; preferably, v is 2;
p is an integer from 1 to 100, preferably 1 to 50; more preferably 1 to 25; most preferably 1 to 10;
A is

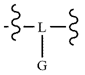

wherein, L is S, O or N; and G, in each instance, is independently, hydrogen, or is taken together with L to form a nitric oxide donor;
X is hydrogen, C₁₋₅ alkyl or is taken together with N to form a nitric oxide donor;
B is hydrogen or —Y—Z, wherein Y is a spacer and Z is a polymer or a terminus group; or B is absent;
D is —NRₐR_{b}, wherein Rₐ and R_{b} are independently selected from the group consisting of hydrogen, formyl, C₁₋₅ alkyl(C═O)—, when the C₁₋₅ alkyl is methyl, Me(C═O)— is an acyl, Ac, C₁₋₅ alkyl and C₁₋₅ alkyl ester;
or D is

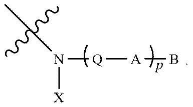

Useful values of R₁, R₂, R₃ and R₄, if present, are each independently selected from the group consisting of hydrogen and C₁₋₅ alkyl. When one or more of R₁, R₂, R₃ and R₄ is C₁₋₅ alkyl, it is selected from methyl, ethyl, n-propyl, isopropyl, t-butyl, n-butyl, isobutyl and pentyl. Preferably, R₁, R₂, R₃ and R₄, if present, is hydrogen or methyl. Most preferably, R₁, R₂, R₃ and R₄, if present, is hydrogen.

In all embodiments,

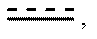

in each instance, is a single or double bond. Preferably, it is a single bond in all instances.

Q is —(CR_{c}R_{d})ᵥ—; wherein R_{c} and R_{d} are independently hydrogen or C₁₋₅ alkyl, such as methyl, ethyl, n-propyl, isopropyl, t-butyl, n-butyl, isobutyl and pentyl. Preferably, R_{c} and R_{d} are independently hydrogen, methyl or ethyl. Useful values of v are integers from 2 to 6. Preferably, v is 2.

Useful values of p include any integer from 1 to 100. Preferably p is an integer from 1 to 50. More preferably, p is an integer from 1 to 25. Most preferably, p is an integer from 1 to 10, such as, 1, 2, 3, 4, 5, 6, 7, 8, 9 and 10.

Useful values of L are N, S and O. Preferably, L is N or S. In each instance that G occurs, it is independently hydrogen or a nitric oxide donor. Since the nitric oxide donor contributes to the amount of available NO on the COS/NO, it is preferable that G is a nitric oxide donor. In preferred embodiments, at least 30% of G present on a COS/NO is a nitric oxide donor. More preferably, at least 50% of G present on a COS/NO is a nitric oxide donor. Even more preferably, at least 90% of G present on a COS/NO is a nitric oxide donor. Most preferably, at least 95% of G present on a COS/NO is a nitric oxide donor.

Useful values of X are hydrogen, C₁₋₅ alkyl or a nitric oxide donor. Since the nitric oxide donor contributes to the amount of available NO on the COS/NO, it is preferable that X is a nitric oxide donor. In preferred embodiments, at least 30% of X present on a COS/NO is a nitric oxide donor. More preferably, at least 50% of X present on a COS/NO is a nitric oxide donor. Even more preferably, at least 90% of X present on a COS/NO is a nitric oxide donor. Most preferably, at least 95% of X present on a COS/NO is a nitric oxide donor.

Useful values of B are hydrogen, —Y—Z, wherein Y is a spacer and Z is a monomer or polymer, or B is a terminus group. B may also be absent when L is O or S. As used herein, a terminus group is any end-capping group at the terminus of a polymer or monomer. These groups are known in the art. Preferably, when B is a terminus group, it is hydrogen, hydroxyl or C₁₋₅ alkyl.

Useful values of Z include monomers and polymers known in the art, especially those used in active pharmaceutical ingredients. Particularly useful polymers or monomers include:

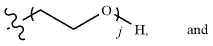

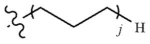

wherein j, in each instance, is an integer from 1 to 100.

Useful spacers, Y, in the formulae disclosed herein include spacers or linkers known in the art, especially those used in active pharmaceutical ingredients. Particularly useful spacers include the following:

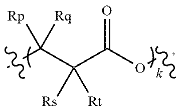

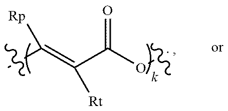

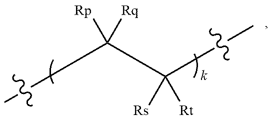

wherein, Rₚ, R_{q}, and Rₜ, in each instance, are independently, hydrogen or hydroxyl; and k is an integer from 1 to 20.

Using the strategies disclosed herein, any secondary amino group present on the oligosaccharide can be modified as described herein to form a NO-releasing oligosaccharide. The secondary amino groups attached directly to the sugar backbone moieties or secondary amino groups pendant on the backbone sugar moieties can be functionalized with a NO releasing moiety.

Useful NO releasing moieties include any NO releasing group known in the art. Particularly useful are residues of NO releasing groups, i.e. NO donors, are covalently bound to N, S or O on the COS/NO. The NO donor is taken together with the atom on the COS/NO to which it is bound to form a moiety selected from the group consisting of a diazeniumdiolate, —NO as part of a nitrosothiol group for example, a nitrosamine, a hydroxyl nitrosamine, a hydroxyl amine, a hydroxyurea, and combination thereof. Preferably, the NO releasing moiety is a diazeniumdiolate. These groups may be present in the form of a salt.

In some embodiments, the NO donor is a N-diazeniumdiolate (i.e., a 1-amino-substituted deazen-1-lum-1,2-diolate) represented by the following:

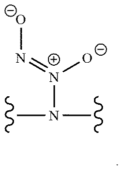

In several embodiments, NXQB is represented by one of the following:

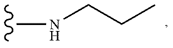

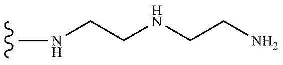

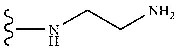

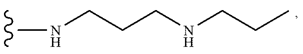

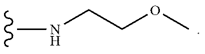

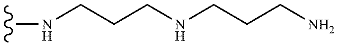

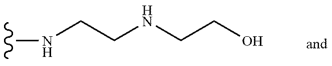

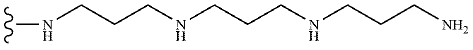

In several embodiments, NXQAB is represented by the following:

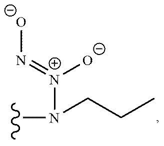

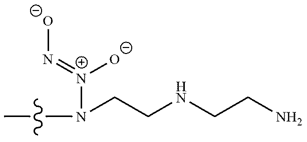

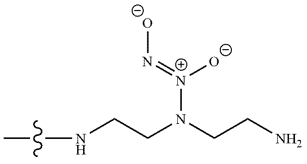

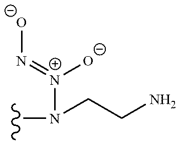

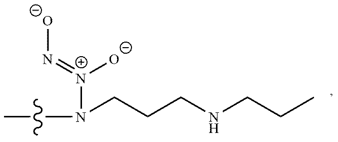

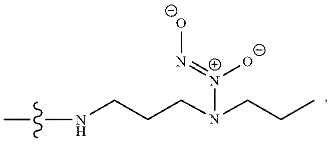

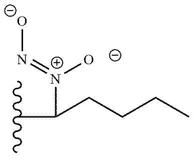 [SMILES: *C(CCCC)/[N+]([O-])=N/[O-]]

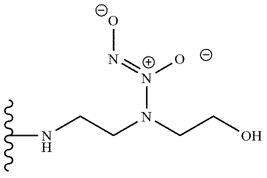 [SMILES: *NCCN(CCO)/[N+]([O-])=N/[O-]]

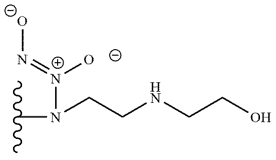 [SMILES: *N(CCNCCO)/[N+]([O-])=N/[O-]]

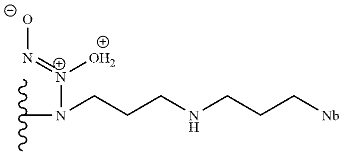

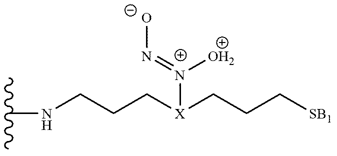

In several embodiments, the COS/NO is represented by the following:

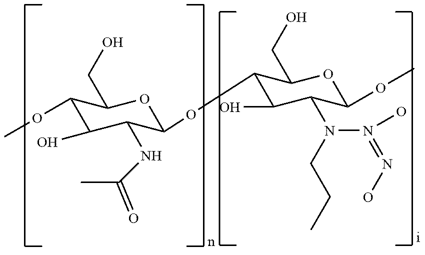

In several embodiments, the COS/NO has a total releasable nitric oxide storage of at least 0.5 µmol of NO per milligram of COS/NO. In several embodiments, the COS/NO has a total releasable nitric oxide storage in a range of about 0.5 µmol to 2.5 µmol of NO per milligram of COS/NO. In several embodiments, greater per milligram NO release is achieved, for example, at least about 2.5 µmol, about 3.0 µmol, about 3.5 µmol, about 4.0 µmol, about 4.5 mol, about 5 µmol or greater amounts of NO per milligram of COS/NO. In several embodiments, the COS/NO has a half-life for nitric oxide release in a range of between about 0.7-4.2 hours. In several embodiments, longer half-lives are achieved, such as for example, about 5 hours, about 6 hours, about 8 hours, about 10 hours, or any time between the listed times. In several embodiments, the COS/NO has a total NO release after 4 hours in a range of between about 0.1-4.0 µmol of NO per milligram of the COS/NO, including about 0.3-2.0 µmol of NO per milligram of the COS/NO, about 0.1-3.0 µmol of NO per milligram of the COS/NO, about 1.5-4 µmol of NO per milligram of the COS/NO, or, about 0.7- 3.0 µmol of NO per milligram of the COS/NO (or any range there between, including endpoints).

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows that nitric oxide increases cell permeability. (a) COS/NO exerts nitrosative and oxidative stress on the cell membrane through the production of multiple reactive byproducts. A more permeable cell membrane will allow for improved diffusion of antibiotics and other hydrophobic molecules. (b) Fluorescent dye NPN fluorescence after exposure to PBS (solid bars), COS/NO at 1 mg/mL (striped bars), COS/NO at 4 mg/mL (stippled bars), or 25% DMSO (checkerboard bars). (c) Fluorescent dye NPN fluorescence in P. aeruginosa strain K after exposure to tobramycin or colistin.

FIG. 2 shows that combinations of COS/NO and antibiotics result in synergy for susceptible strains of P. aeruginosa. (a) 24 h time kill assay of PAK exposed to combinations of COS/NO and tobramycin (solid bars) or COS/NO and colistin (striped bars). (b) 24 h time kill assay with MDR strains of P. aeruginosa exposed to COS/NO (solid bars), tobramycin (striped bars), COS/NO and tobramycin (stippled bars), or PBS (checkerboard bars).

FIG. 3 shows PAK viability after exposure to antibiotics without (solid line) or with NO-pretreatment at 25% (dashed line) or 100% (dotted line) COS/NO MIC for 4 h. Error is representative of the standard deviation of the mean for ≥3 biological replicates.

FIG. 4 shows that nitric oxide pretreatment of P. aeruginosa biofilms results in improved tobramycin susceptibility. (a) PAK biofilm viability after exposure to tobramycin without pretreatment (solid bars) or with NO pretreatment of 1 h (striped bars), 2 h (stippled bars), or 4 h (checkerboard bars). (b-e) MDR P. aeruginosa biofilm viability after exposure to tobramycin without pretreatment (solid bars) or with NO pretreatment of 4 h (striped bars). Error is representative of the standard deviation of the mean for ≥3 biological replicates.

FIG. 5 shows that serial exposure to sub-inhibitory doses of NO does not result in any change in MIC for PAK or ATCC MRSA. (a) The MIC of PAK for tobramycin (solid line), NO (long dash), or tobramycin when delivered concurrently with COS/NO at 25% of its MIC (short dash). (b) The MIC of ATCC MRSA for tobramycin (solid line), NO (long dash), or tobramycin when delivered concurrently with COS/NO at 25% of its MIC (short dash). NO doses from COS/NO were determined by chemiluminescence.

FIG. 6 shows NO-release kinetics and totals of COS/NO in PBS pH 7.4. (a) Instantaneous flux of NO release. (b) Total NO released over time. Error is representative of the standard deviation of the mean of n≥3 separate measurements.

FIG. 7 shows representative ¹H NMR of COS/NO in deuterium oxide.

FIG. 8 shows representative FT-IR of 5 kDa chitosan (blue) and COS/NO (red).

FIG. 9 shows that nitric oxide pretreatment of MDR species of P. aeruginosa results in improved tobramycin susceptibility. (a-d) P. aeruginosa viability after exposure to tobramycin with (dashed line) and without (solid line) NO pretreatment. Error is representative of the standard deviation of the mean for ≥3 biological replicates.

FIG. 10 shows that that nitric oxide pretreatment of MDR ESKAPE pathogens results in improved tobramycin susceptibility. (a-d) ESKAPE pathogen viability after exposure to tobramycin with (dashed line) and without (solid line) NO pretreatment. Error is representative of the standard deviation of the mean for ≥3 biological replicates.

FIG. 11 shows that nitric oxide pretreatment of P. aeruginosa biofilms results in improved tobramycin susceptibility. (a) PAK biofilm viability after exposure to tobramycin without pretreatment (solid bars) or with NO pretreatment of 1 h (striped bars), 2 h (stippled bars), or 4 h (checkerboard bars). (b-e) MDR P. aeruginosa biofilm viability after exposure to tobramycin without pretreatment (solid bars) or with NO pretreatment of 4 h (striped bars). Error is representative of the standard deviation of the mean for ≥3 biological replicates.

FIG. 12 shows that NO-pretreatment of P. aeruginosa biofilms does not significantly reduce viability. Error is representative of the standard deviation of the mean of n≥3 separate measurements.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

### Materials & Methods

### Materials

Medium molecular weight chitosan was purchased from Primex (Siglufjordur Iceland). Potassium nitrate, HEPES, tobramycin, ethanol amine, p-anisaldehyde, and p-toluenesulfonyl chloride were purchased from Millipore Sigma (St. Louis, MO). Argon, nitrogen (N₂), nitric oxide (NO) calibration (25.87 ppm, balance N₂) and pure NO (99.5%) gas cylinders were purchased from Airgas National Welders (Raleigh, NC). Distilled water was purified using a Millipore Milli-Q UV Gradient A10 System (Bedford, MA) to a resistivity of 18.2 MΩ·cm and a total organic content of ≤ 10 ppb.

### Bacteria Strains and Media

The laboratory Pseudomonas aeruginosa strain K (PAK) was donated by Professor Matthew Wolfgang from the Department of Microbiology and Immunology at the University of North Carolina at Chapel Hill (Chapel Hill, NC). Staphylococcus aureus (ATCC #29213), Methicillin-resistant S. aureus (MRSA, ATCC #33591) and Burkholderia cepacia complex (BCC, ATCC #25416) were obtained from the American Type Tissue Culture Collection (Manassas, VA). Clinical multidrug resistant strains of P. aeruginosa (AR 229, AR 230, AR 237, and AR 239), and Klebsiella pneumoniae (AR 542) were obtained from the CDC & FDA Antibiotic Resistant Isolate Bank (Atlanta, GA). Tryptic soy broth (TSB) and tryptic soy agar (TSA) were obtained from Becton, Dickinson and Company (Franklin Lakes, NJ).

### Synthesis of Nitric Oxide-Releasing Chitosan Oligosaccharides

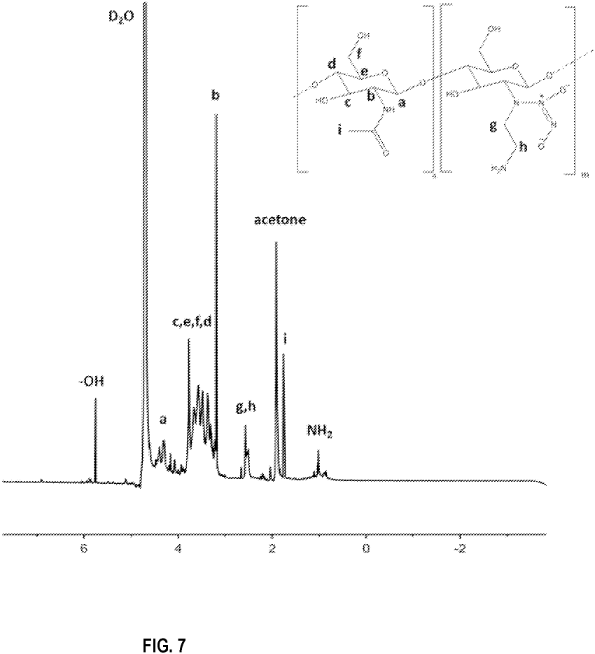

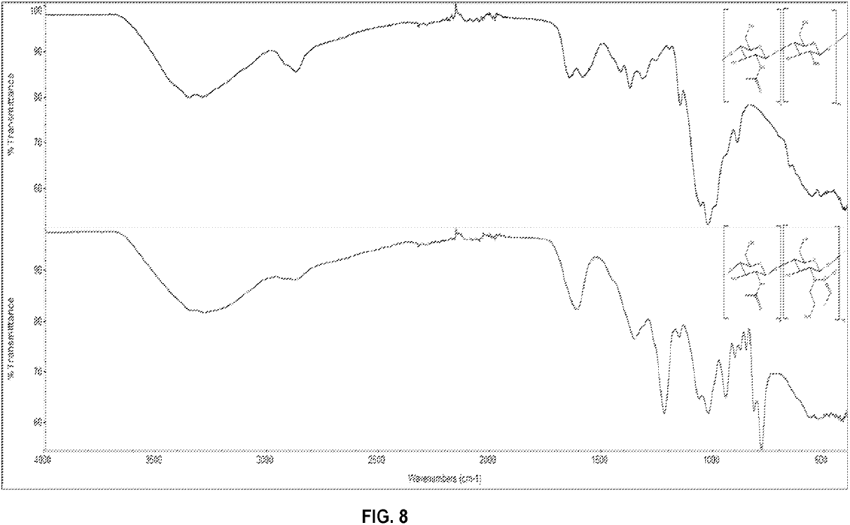

Secondary amine-modified chitosan oligosaccharides were prepared as previously described [Ahonen, M. J. R., Hill, D. B. & Schoenfisch, M. H. Nitric oxide-releasing alginates as mucolytic agents. ACS Biomater. Sci. Eng. 5, 3409-3418 (2019)]. Briefly, medium molecular weight chitosan (5 g) was oxidatively degraded in hydrogen peroxide (100 mL, 15%) at 85° C. for 1 h. The resulting solution was filtered to remove insoluble components, and chitosan oligosaccharides were precipitated with ethanol, collected via centrifugation, and dried in vacuo. Chitosan oligosaccharides were subsequently modified with an aminoethyl Schiff base functional group through a tosylated nucleophilic substitution reaction to produce secondary amine-modified chitosan oligosaccharides (COS). Amine-modified chitosan (45 mg) was dissolved in a mixture of water (450 µL), methanol (2.55 mL), and sodium methoxide (5.4 mM in methanol, 75 µL) and subsequently placed into a Parr hydrogenation vessel with continuous stirring. Oxygen was removed with three short argon purges (10 s, 7 bar) followed by three long argon purges (10 min, 7 bar). The reactor was pressurized with NO gas (10 bar) for 3 days. The same purging procedure was performed to removal unreacted NO. The NO-releasing chitosan oligosaccharides (COS/NO) were precipitated in ethanol, collected via centrifugation, dried in vacuo, and stored at - 20° C. in vacuum sealed bags. Successful synthesis was confirmed with NMR and FTIR, and compared to previously published literature [Ahonen, M. J. R., Hill, D. B. & Schoenfisch, M. H. Nitric oxide-releasing alginates as mucolytic agents. ACS Biomater. Sci. Eng. 5, 3409-3418 (2019)]. Representative data are provided in Supporting Information (FIG. 7 and FIG. 8).

### Nitric Oxide-Release Kinetics and Totals

Nitric oxide-release was measured in real time with a Zysense chemiluminescent nitric oxide analyzer (NOA, Boulder, CO). Approximately 1 mg of COS/NO was added into 30 mL PBS (10 mM, pH 7.4) and carried to the instrument via nitrogen bubbled through solution at 200 mL/min. Analysis was stopped when measurements fell below 10 ppb NO per mg of COS.

### Single Agent Minimum Inhibitory Assays

Frozen stocks of bacteria were reconstituted in TSB (3 mL) and cultured overnight. The overnight cultures of bacteria (3 mL) were inoculated into fresh TSB (30 mL) and grown to 10⁸ CFU/mL. Bacteria were diluted in TSB to a final concentration of 10⁶ CFU/mL and exposed to serial dilutions of COS/NO or antibiotic (aztreonam, ceftazidime, ciprofloxacin, colistin, meropenem, and tobramycin) for 24 h. Inhibition was assessed with the resazurin assay and the minimum inhibitory concentration (MIC) was defined as the lowest concentration of antibacterial agent required to prevent the reduction of resazurin (i.e., color change from blue to pink).

### Checkerboard Assays

The checkerboard method was employed as previously described to experimentally determine the efficacy of COS/NO and each antibiotic (aztreonam, ceftazidime, ciprofloxacin, colistin, meropenem, and tobramycin) in combination [Privett, B. J. et al. Synergy of nitric oxide and silver sulfadiazine against gram-negative, gram-positive, and antibiotic-resistant pathogens. Mol. Pharm. 7, 2289-2296 (2010)]. Briefly, bacteria at a final concentration of 10⁶ CFU/mL were incubated with an array of antimicrobial combinations in TSB for 24 h at 37° C. The highest concentration for each antimicrobial tested was 2 × MIC. Six additional dosages at stepwise, 2-fold dilutions in concentration were evaluated, resulting in 49 total combinations of COS/NO and each antibiotic tested against each strain of bacteria. The lowest drug concentration in the array that did not support bacterial growth nor change color after incubation with resazurin was determined the most effective inhibitory concentration. The fractional bactericidal concentration index (ΣFIC) was calculated using Equation 1, reported by Elion et al. [Elion, G. B., Singer, S. & Hitchings, G. H. Antagonists of Nucleic Acid Derivatives: VIII. Synergism in combinations of biochemically related antimetabolites. J. Biol. Chem. 208, 477-488 (1954)] where MIC_{A} and MIC_{B} are the values determined for agents A and B in the single-agent assays, respectively, and MIC_{AB} and MIC_{BA} are the concentrations of agent A and B that constituted the most effective inhibitory combination as determined by the checkerboard assay. Checkerboard assays were conducted in at least duplicate for each bacterial strain and for each drug combination.

$$Σ \text{FIC=} \frac{{\text{MIC}}_{\text{AB}}}{{\text{MIC}}_{\text{A}}} + \frac{{\text{MIC}}_{\text{BA}}}{{\text{MIC}}_{\text{B}}}$$

Characterization of the combinations was performed using the following criteria based on ΣFIC values: < 0.25 is highly synergistic; ≤ 0.5 is synergistic; ≤ 1 is additive; ≤ 4 is indifferent; > 4 is antagonistic.

### Combination Time Kill Assays

Time-kill assays were performed over 24 h in order to quantitatively probe the effect of each antibiotic-NO combination as a function of time [Belley, A. et al. Assessment by time-kill methodology of the synergistic effects of oritavancin in combination with other antimicrobial agents against Staphylococcus aureus. Antimicrob. Agents Chemother. 52, 3820-3822 (2008)]. Planktonic bacteria at a final concentration of 10⁶ CFU/mL in TSB were incubated with combinations of COS/NO and antibiotic at 1 × MIC. Aliquots were sampled at 0, 3, 6, and 24 h, serially diluted, and logarithmically plated on TSA plates using an Eddy Jet spiral plater (IUL; Farmingdale, NY). Bacterial viability was assessed by counting the number of colonies formed with a Flash & Go colony counter (IUL; Farmingdale, NY). Synergy was defined as showing a 2-log decrease in colony count after 24 h by the combination compared to that by the most active single agent alone, and antagonism as showing a 2-log increase in colony count after 24 h by the combination compared to the most active single agent [Belley, A. et al. (2008)] Indifference was defined as a less than 2-log increase or decrease by combination compared to the most active single agent.

### Nitric Oxide Pretreatment with Planktonic Bacteria

Bacteria were cultured and diluted to a final concentration of 10⁶ CFU/mL in TSB as described previously and incubated with subinhibitory concentrations (¼ x MIC) of COS/NO for 1, 2, or 4 h in a 1-dram vial, added to serial dilutions of antibiotic (i.e., aztreonam, colistin, meropenem, or tobramycin), and incubated for 20 h. After exposure, wells were serially diluted in sterile MilliQ water and spiral plated on TSA. Viability was assessed by colony counting.

For combination assays, bacteria at a final concentration of 10⁶ CFU/mL in TSB were incubated with subinhibitory concentrations (¼ x MIC) of COS/NO for 4 h in a 15 mL centrifuge tube and subsequently incubated with an array of antibiotic combinations in TSB for 24 h at 37° C. The highest antibiotic concentration tested was 2 × MIC. Six additional dosages at stepwise, 2-fold dilutions in concentration were evaluated, resulting in 49 total combinations of tobramycin and either colistin or aztreonam tested against each strain of P. aeruginosa. The lowest drug concentration in the array that did not support bacterial growth nor change color after incubation with resazurin was determined the most effective inhibitory concentration. The ΣFIC was calculated using Equation 1. Checkerboard assays were conducted in at least duplicate for each strain and characterized using the previously described criteria.

### NPN Uptake Assay

The outer membrane permeability of PAK, ATCC MRSA, ATCC BCC, and AR 542 was assessed with an NPN assay modified from Helander and coworkers [Helander, I. M. & Mattila-Sandholm, T. Fluorometric assessment of Gram-negative bacterial permeabilization. J. Appl. Microbiol. 88, 213-219 (2000)]. Briefly, a stock 4560 µM NPN in acetone solution was prepared fresh each day and diluted to 456 µM in PBS pH 7.4. Bacteria were cultured to a concentration of 10⁸ CFU/mL in TSB, collected via centrifugation (5,000 × g for 5 min), and resuspended in PBS. Bacteria were added to COS/NO or DMSO in 1-dram vials and incubated at 37° C. with shaking. After 10 min of incubation, a portion of the exposure solution (196 µL) was removed and added to a black well plate, and 4 µL NPN (456 µM in PBS) was added. Fluorescence was immediately measured at excitation and emission wavelengths of 350 nm and 460 nm, respectively. The background NPN fluorescence in buffer was subtracted.

### Minimum Biofilm Eradication Concentration Assays

Bacteria were cultured to a concentration of 10⁸ CFU/mL in TSB and diluted to 10⁶ CFU/mL in 200 µL TSB in a 96 well plate. Plates were incubated with shaking for 3 days until a non-surface attached viscous aggregate formed that was easily separated from growth medium. Biofilms (100 µL) were removed, gently injected into PBS (200 µL) to remove loosely adhered planktonic cells, and added to a sterile 96 well plate. To the wells, PBS or test agent dissolved in PBS (100 µL) was added and incubated with shaking for 24 h. The biofilm (100 µL) was removed from the well plate, added to 900 µL sterile MilliQ water, and disrupted with pipetting and vortexing. Disrupted biofilms were serially diluted and spiral plated on TSA. Viability was assessed with colony counting. The minimum biofilm eradication concentration (MBEC) was defined as the lowest concentration of test agent required to reduce viability by 5-log (i.e., 10⁸ to 10³ CFU/mL).

### Biofilm Pretreatment with Nitric Oxide

Biofilms were grown as described previously and exposed to COS/NO at ¼ x MBEC for 1, 2, or 4 h. The biofilm was then removed from its 96 well plate, added to a new 96 well plate containing tobramycin dissolved in PBS (100 µL), and incubated for 20 h. The biofilm was disrupted in 900 µL sterile MilliQ water with pipetting and vortexing, serially diluted, and spiral plated on TSA. Viability was assessed with colony counting.

### Serial Passaging Resistance Assays

Bacteria at a final concentration of 10⁶ CFU/mL in TSB were incubated with serial dilutions of COS/NO and/or tobramycin for 24 h. Growth was assessed by measuring absorbance at 600 nm. The wells that had the highest concentration of test agent and an OD₆₀₀ corresponding to more than 10⁸ CFU/mL were diluted to 10⁶ cfu/mL in TSB and incubated with fresh solutions of COS/NO or tobramycin for 24 h. A single passage was defined as one exposure, incubation, and subsequent dilution. Bacteria were passaged for up to 70 days.

### Examples

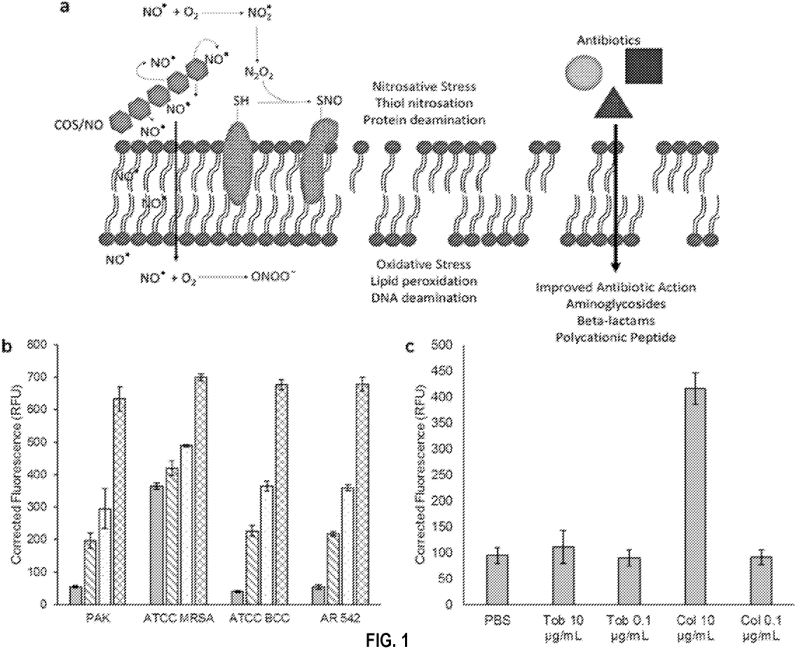

Nitric oxide represents a potential solution to the threat of antibiotic resistance. As a diatomic radical, NO rapidly generates several reactive oxygen and nitrogen species in physiological conditions that kill bacteria through multiple mechanisms (e.g., lipid peroxidation, protein deamination, FIG. 1, panel A) [Hetrick, E. M. et al. Bactericidal efficacy of nitric oxide-releasing silica nanoparticles. ACS Nano 2, 235-246 (2008); Wink, D. A. et al. DNA deaminating ability and genotoxicity of nitric oxide and its progenitors. Science (80-. ). 254, 1001-1003 (1991); Barraud, N., Kelso, M., Rice, S. & Kjelleberg, S. Nitric Oxide: A Key Mediator of Biofilm Dispersal with Applications in Infectious Diseases. Curr. Pharm. Des. 21, 31-42 (2014)]. One of the major mechanisms through which NO exerts bactericidal action through compromising the cell membrane and increasing permeability which others have shown with atomic force microscopy and confocal microscopy [Hetrick, E. M. et al. Bactericidal efficacy of nitric oxide-releasing silica nanoparticles. ACS Nano 2, 235-246 (2008); Deupree, S. M. & Schoenfisch, M. H. Morphological analysis of the antimicrobial action of nitric oxide on Gram-negative pathogens using atomic force microscopy. Acta Biomater. 5, 1405-1415 (2009)], and the applicants demonstrate herein by an increased uptake of the hydrophobic fluorophore NPN and crystal violet dye (FIG. 1, panels B,C). Membrane damage allows for compounds that normally cannot penetrate the membrane to cross more readily, and NO-mediated membrane damage may allow for increased diffusion of antibiotics (FIG. 1, panel A). Previous work has demonstrated that multiple cases of synergism are attributable to increased cell permeability and antibiotic uptake [Bollenbach, T. Antimicrobial interactions: mechanisms and implications for drug discovery and resistance evolution. Curr. Opin. Microbiol. 27, 1-9 (2015); Khalil, H., Chen, T., Riffon, R., Wang, R. & Wang, Z. Synergy between Polyethylenimine and Different Families of Antibiotics against a Resistant Clinical Isolate of Pseudomonas aeruginosa. Antimicrob. Agents Chemother. 52, 1635-1641 (2008)].

Thus, ESKAPE pathogen susceptibility to antibiotics may improve by harnessing the disruptive capabilities of NO, which simultaneously exerts bactericidal action. Because NO exists naturally as a highly reactive gas, the applicants deliver NO into solution via a water-soluble chitosan donor that releases NO under physiological conditions in a burst release profile (FIG. 6 and Table 1). Nitric oxide-releasing chitosan (COS/NO) has been used previously by our lab to eradicate both planktonic bacteria and biofilms [Lu, Y., Slomberg, D. L. & Schoenfisch, M. H. Nitric Oxide-Releasing Chitosan Oligosaccharides as Antibacterial Agents. Biomaterials 35, 1716-1724 (2014); Reighard, K. P. & Schoenfisch, M. H. Antibacterial action of nitric oxide-releasing chitosan oligosaccharides against Pseudomonas aeruginosa under aerobic and anaerobic conditions. Antimicrob. Agents Chemother. 59, 6506-6513 (2015)], and COS/NO outperforms NO gas as a bactericidal agent [Hall, J. R. et al. Mode of nitric oxide delivery affects antibacterial action. ACS Biomater. Sci. Eng. acsbiomaterials.9b01384 (2019)].

N-diazeniumdiolate NO donor ligands are employed to facilitate NO storage and release from chemically modified chitosan oligosaccharides (COS/NO). Additional ligands are disclosed in U.S. Pat. Nos. 98503222 and 10759877, incorporated by reference herein. The chitosan biopolymer releases two molecules of NO per secondary amine upon breakdown of the N-diazeniumdiolate at physiological pH (i.e., protonation, FIG. 7), with the NO exerting broad-spectrum bactericidal action.

TABLE 1

NO-release from COS/NO in PBS pH 7.4 determined via chemiluminescence

| [NO]ₜ^{a}(µmol/mg) | [NO]ₘₐₓ^{b}(pmol/mg) | t_{½}^{c}(h) | t_{d}^{d}(h) | [NO]₄ₕ^{e}(µmol/mg) |
|---|---|---|---|---|
| 0.80 ± 0.10 | 450 ± 35 | 7.0 ± 1.1 | 30.1 ± 4.6 | 0.31 ± 0.06 |

^{a} Total NO released. ^{b} Maximum flux of NO release. ^{c} NO-release half-life. ^{d} Duration of NO-release. ^{e}Total NO released after 4 h.

### Combinations of Nitric Oxide and Antibiotics

The effects of antibiotic-COS/NO combinations were investigated first in planktonic P. aeruginosa strain K (PAK) using classic checkerboard assays [Caleffi-Ferracioli, K. R., Maltempe, F. G., Siqueira, V. L. D. & Cardoso, R. F. Fast detection of drug interaction in Mycobacterium tuberculosis by a checkerboard resazurin method. Tuberculosis 93, 660-663 (2013); Privett, B. J. et al. Synergy of nitric oxide and silver sulfadiazine against gram-negative, gram-positive, and antibiotic-resistant pathogens. Mol. Pharm. 7, 2289-2296 (2010)]. Checkerboard assays characterize antimicrobial combinations as synergistic, additive, indifferent, or antagonistic using fractional inhibitory concentration indices (ΣFIC). ΣFIC values are calculated using an equation that evaluates the inhibitory action of each test agent in the combination and alone, and when the effects of the combination are greater than the sum of the individual agents, the result in synergy. The opposite effect, antagonism, occurs when the effects of the combination are worse than the individual agents, which is dangerous and undesirable for clinical applications.

No antagonism was observed in any of the six tested antibiotic-NO combinations, and most where classified as either synergistic or additive interactions (Table 2). These data indicate that NO may be used in combination with a variety of antibiotic classes and is unlikely to interfere with the antibiotic mechanism of action. Observations from checkerboard assays were supported by quantitative data obtained in 24 h time kill assays, where COS/NO and either tobramycin or colistin were combined at their respective minimum inhibitory concentrations (MIC) for 24 h [Ciacci, N. et al. In vitro Synergism of Colistin and N-acetylcysteine against Stenotrophomonas maltophilia. Antibiotics 8, 101 (2019)]. By quantifying viability after exposure, the interactions between COS/NO and the antibiotic can be characterized as synergistic, indifferent, or antagonistic. Both colistin- and tobramycin-NO combinations resulted in ≥2-log decrease in viability compared to the most active single agent, which is defined as synergy. Thus, antibiotic-NO combinations were more effective together than as single agents against PAK.

TABLE 2

Median fractional inhibitory concentration indices for antibiotic combinations with COS/NO against PAK. Data is representative of ≥3 biological replicates

| Antibiotic | Antibiotic Class | Mechanism of Action | ΣFIC | Classification |
|---|---|---|---|---|
| Aztreonam | Beta-lactam | Inhibits cell wall synthesis | 0.75 | Additive |
| Ceftazidime | Cephalosporin | Inhibits cell wall synthesis | 1 | Additive |
| Ciprofloxacin | Fluoroquinolone | Inhibits DNA replication | 1.25 | Indifferent |
| Colistin | Polycationic peptide | Disrupts outer membrane | 0.5 | Synergistic |
| Meropenem | Beta-lactam | Inhibits cell wall synthesis | 0.5 | Synergistic |
| Tobramycin | Aminoglycoside | Prevents mRNA translation | 0.625 | Additive |

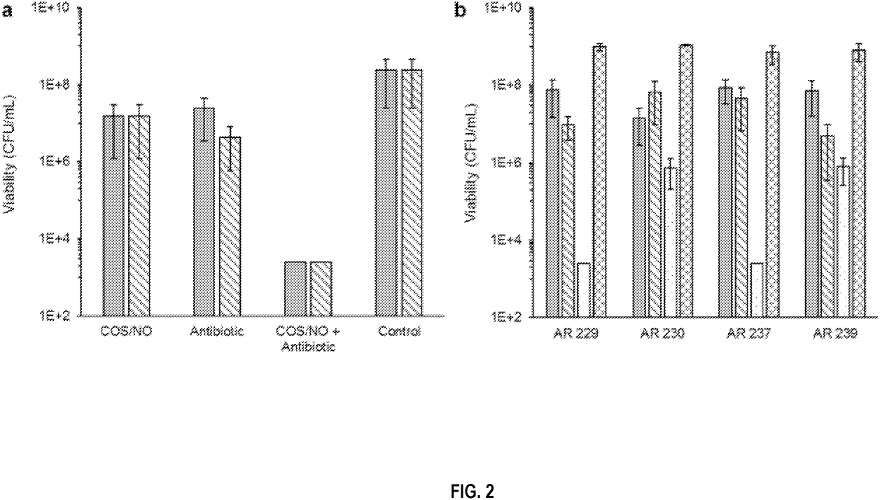

To more rigorously evaluate COS/NO as a potential combination therapy, multidrug resistant P. aeruginosa isolates obtained from the U.S. Centers for Disease Control were examined. Two strains (PA 229 and PA 237) are susceptible to tobramycin while two others (PA 230 and PA 239) are tobramycin-resistant and possess multiple genes for aminoglycoside modification enzymes (Table 3). The tobramycin-susceptible strains (PA 229 and PA 237) were killed synergistically with tobramycin-NO combinations while interactions between tobramycin and NO were indifferent in tobramycin-resistant strains (FIG. 2). The bactericidal action by tobramycin was not significantly improved with the simultaneous addition of NO likely due to deactivation via aminoglycoside modification enzymes. Nevertheless, no antibiotic-NO combination resulted in antagonism; all interactions were synergistic, additive, or indifferent, even in MDR P. aeruginosa strains.

TABLE 3

Molecular mechanisms of resistance for MDR P. aeruginosa isolates ^{a}

| | | |
|---|---|---|
| Strain | Antibiotic Class | Gene |
| PA 229 | Beta-lactam | OXA-50, PAO |
| PA 230 | Aminoglycoside | aax(3)-ld, aadA2 |
| | Beta-lactam | OXA-4, OXA-50, PAI, VIM-2 |
| | Phenicols | cmlA 1 |
| | Tetracyclines | tet(G) |
| | Trimethoprim | dfrB5 |
| PA 237 | Beta-lactam | OXA-50, PAO |
| | Phenicols | catB7 |
| PA 239 | Aminoglycoside | aac(6ʹ)-IIa, aadB, aph(3ʹ)-Ic, strA, strB |
| | Beta-lactam | GES-1, OXA-10, OXA-50, VIM-11 |
| | Phenicols | cmla 1 |
| | Tetracyclines | tet(G) |
| | Trimethoprim | dfrB5 |

^{a} Data from the CDC and FDA Antibiotic Resistance Isolate Bank

### Nitric Oxide Pretreatment

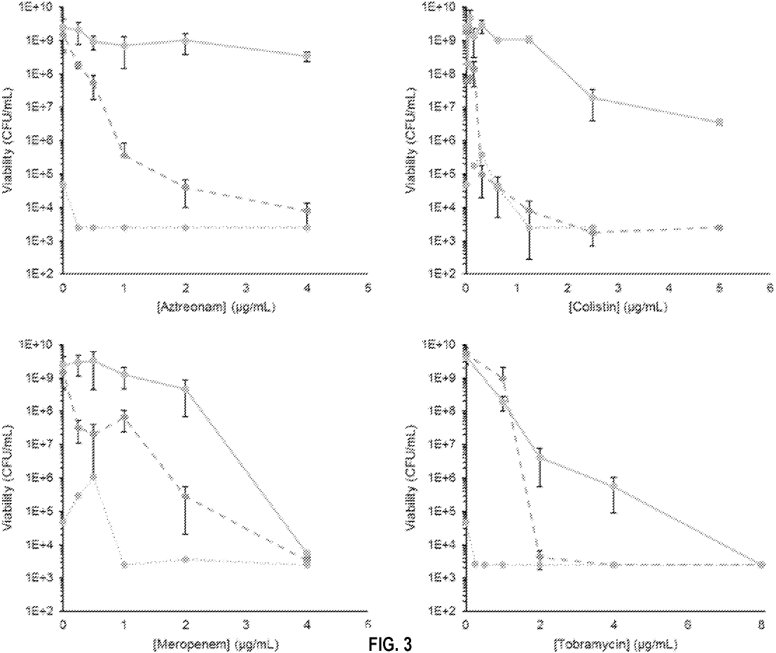

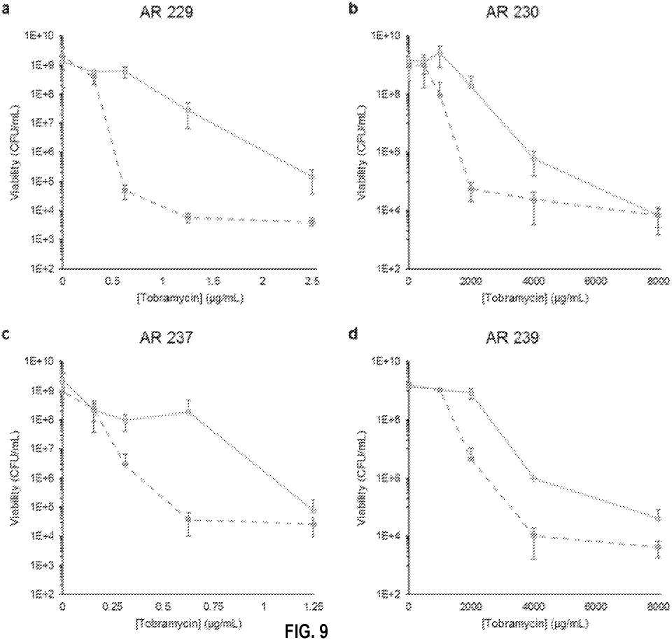

Because NO improves bacterial cell permeability, NO exposure may improve the bactericidal action of antibiotics by decreasing the barrier presented by the cell membrane. Therefore, 24 h time kill assays were modified to include a 4 h exposure to COS/NO prior to the addition of antibiotic. By first exposing P. aeruginosa to low doses of NO (25% of the MIC), the bactericidal action of antibiotics was markedly improved across all tested strains and antibiotics (Table 4). PAK susceptibility to aztreonam, colistin, meropenem, and tobramycin (FIG. 3 and Table 4) decreased by 3- to 5-log with NO-pretreatment compared to without. Additionally, even the multidrug-resistant strains of P. aeruginosa were more susceptible to tobramycin after NO-pretreatment (Table 4 and FIG. 9). Using classification criteria from 24 h time-kill assays, synergistic bactericidal action was achieved with all combinations of NO with antibiotics.

TABLE 4

The log difference in bacteria viability after exposure to antibiotic with or without NO-pretreatment ^{a}

| Strain | Antibiotic | [Antibiotic] (µg/mL) | Log Difference^{b} | Classification^{c} |
|---|---|---|---|---|
| PAK | Aztreonam | 2 | 5.11 | Synergistic |
| | Colistin | 1.25 | 5.13 | Synergistic |
| | Meropenem | 2 | 3.22 | Synergistic |
| | Tobramycin | 2 | 3.00 | Synergistic |
| AR 229 | Tobramycin | 0.625 | 4.08 | Synergistic |
| AR 230 | Tobramycin | 2000 | 3.54 | Synergistic |
| AR 237 | Tobramycin | 0.625 | 3.70 | Synergistic |
| AR 239 | Tobramycin | 2000 | 2.27 | Synergistic |
| ATCC S.aureus | Tobramycin | 125 | 4.88 | Synergistic |
| ATCC MRSA | Tobramycin | 4000 | 4.09 | Synergistic |
| ATCC BCC | Tobramycin | 125 | 5.23 | Synergistic |
| AR 542 | Tobramycin | 500 | 2.29 | Synergistic |

^{a} NO-pretreatment was performed at 25% of the minimum inhibitory concentration of NO for 4 h. ^{b} The log of bacteria viability after exposure to antibiotic with NO-pretreatment subtracted from without NO-pretreatment. ^{c} Classifications: Synergy ≥2-log, Indifferent <2-log, Antagonism >-2-log

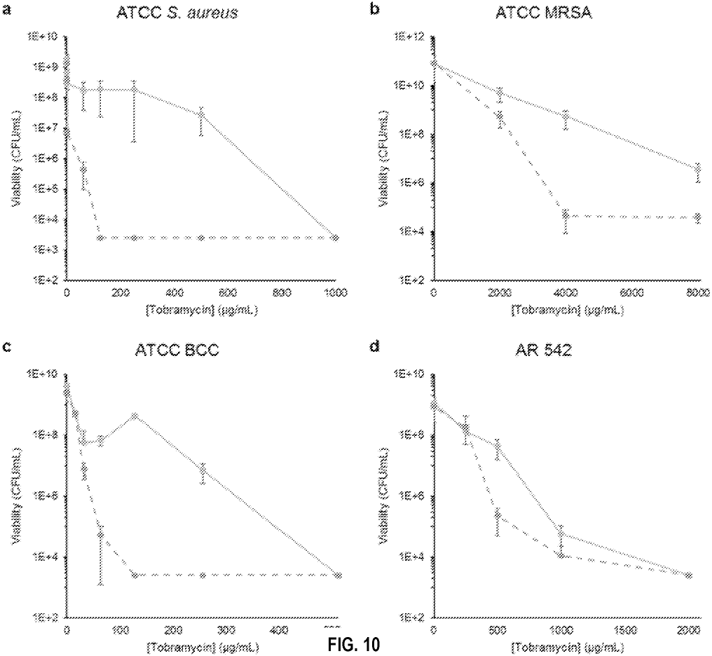

A similar trend was observed in the other tested ESKAPE pathogens (i.e., K. pneumoniae AR 542, ATCC S. aureus, and ATCC MRSA) and the intrinsically resistant B. cepacia complex, where a significant improvement in tobramycin-susceptibility was observed with the addition of NO (Table 4 and FIG. 10). These data suggest that NO can be used to increase antibiotic-susceptibility in a wide variety of multidrug resistant pathogens. Both S. aureus and MRSA susceptibility to tobramycin increased with NO-pretreatment, indicating that the “sensitizing action” of NO was not unique to Gram-negative bacteria. Nitric oxide was able to improve the bactericidal efficacy of all tested antibiotics, regardless of mechanism of action, in both Gram-positive and Gram-negative bacteria, irrespective of resistance profile.

### Nitric Oxide Pretreatment of P. Aeruginosa Biofilms

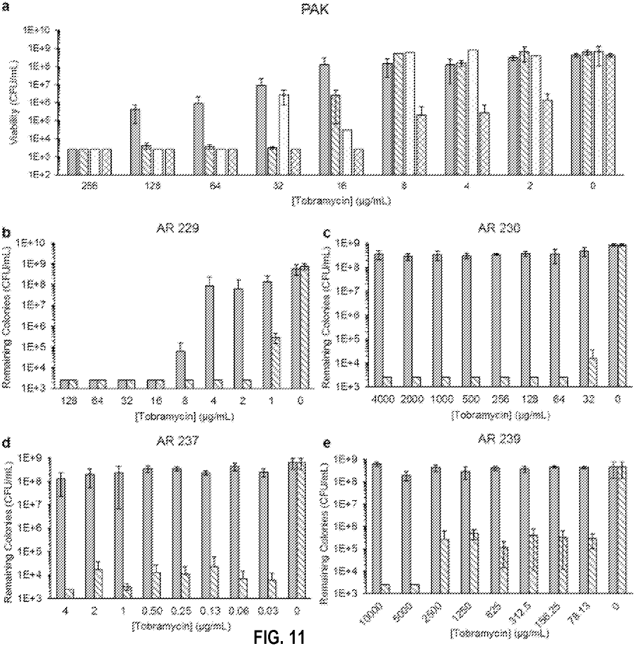

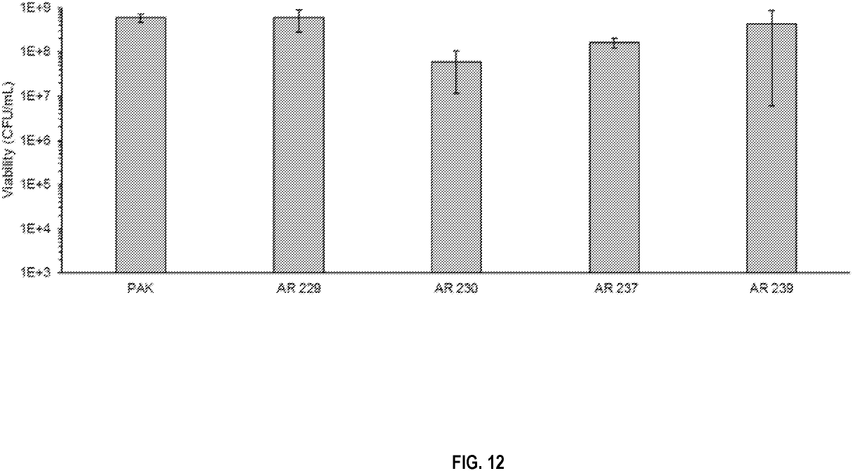

Biofilms are inherently resistant to antibiotic penetration and action due primarily to the protective matrix and altered metabolic state of enclosed bacteria. Nitric oxide has previously been shown to disrupt P. aeruginosa biofilms, and rheological analysis has suggested that the biofilm matrix is compromised with NO treatment [Reighard, K. P., Hill, D. B., Dixon, G. A., Worley, B. V. & Schoenfisch, M. H. Disruption and eradication of P. aeruginosa biofilms using nitric oxide-releasing chitosan oligosaccharides. Biofouling 31, 775-87 (2015); Howlin, R. P. et al. Low-Dose Nitric Oxide as Targeted Anti-biofilm Adjunctive Therapy to Treat Chronic Pseudomonas aeruginosa Infection in Cystic Fibrosis. Mol. Ther. 25, 2104-2116 (2017)]. P. aeruginosa biofilms were grown for 3 days and exposed to COS/NO at 25% of the minimum biofilm eradication concentration (MBEC) for 4 h prior to the addition of tobramycin. Minimal changes in tobramycin-susceptibility were observed with NO-pretreatment lasting less than 4 h (FIG. 11, panel A). However, all tested P. aeruginosa biofilms, including the tobramycin-resistant strains, were significantly more susceptible to tobramycin after NO-pretreatment (FIG. 11 and Table 5). Of note, NO-pretreatment does not significantly affect biofilm viability (FIG. 12). Together the data indicate that NO-pretreatment improves the antibiotic susceptibility of P. aeruginosa strains, including tobramycin-resistant ones.

TABLE 5

The log difference in P. aeruginosa biofilm viability after exposure to tobramycin with or without NO-pretreatment ^{a}

| Strain | [Tobramycin] (µg/mL) | Log Difference ^{b} | Classification ^{c} |
|---|---|---|---|
| PAK | 16 | 4.71 | Synergistic |
| AR 229 | 2 | 4.39 | Synergistic |
| AR 230 | 64 | 5.15 | Synergistic |
| AR 237 | 1 | 4.85 | Synergistic |
| AR 239 | 5000 | 4.90 | Synergistic |

^{a} NO-pretreatment was performed at 25% of the minimum inhibitory concentration of NO for 4 h. ^{b} The log of bacteria viability after exposure to antibiotic with NO-pretreatment subtracted from without NO-pretreatment. ^{c} Classifications: Synergy ≥2-log, Indifferent <2-log, Antagonism >-2-log.

### Serial Passaging with Nitric Oxide

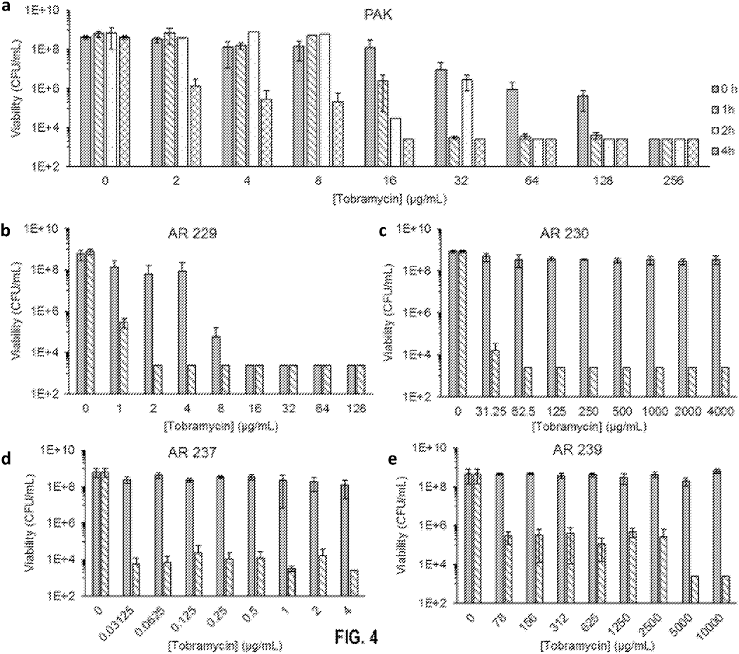

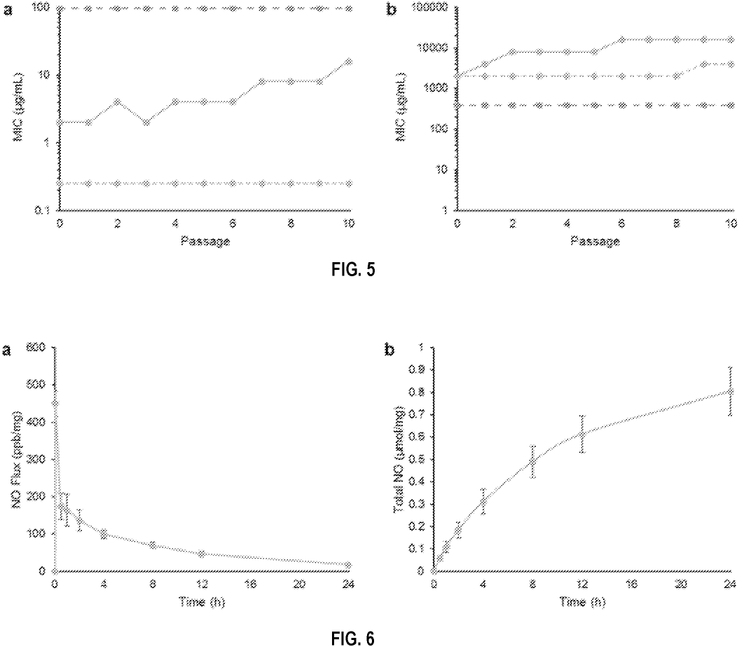

Continuous exposure to subinhibitory concentrations of antibiotics results in the development of resistant bacteria [Spellberg, B. et al. The Epidemic of Antibiotic-Resistant Infections: A Call to Action for the Medical Community from the Infectious Diseases Society of America. Clin. Infect. Dis. 46, 155-164 (2008); Ghosh, S. & LaPara, T. M. The effects of subtherapeutic antibiotic use in farm animals on the proliferation and persistence of antibiotic resistance among soil bacteria. ISME J. 1, 191-203 (2007)] thus concerns about engendering resistance to NO have been expressed. A study performed previously by our lab demonstrated that exogenous NO delivered at subinhibitory concentrations by silica nanoparticles did not result in any phenotypic changes (i.e., MICs) in any tested bacteria [Privett, B. J., Broadnax, A. D., Bauman, S. J., Riccio, D. A. & Schoenfisch, M. H. Examination of Bacterial Resistance to Exogenous Nitric Oxide. Nitric Oxide 26, 126-173 (2012)]. Because previous work demonstrated that the development of resistance depends upon antibiotic exposure parameters [Martinez, J. L. & Baquero, F. Mutation Frequencies and Antibiotic Resistance. Antimicrob. Agents Chemother. 44, 1771-1777 (2000)] the applicants investigated the effects of repeated exposure to COS/NO at subinhibitory concentrations. Over 10 consecutive passages, the MIC of COS/NO for P. aeruginosa and MRSA remained unchanged (FIG. 4). Parallel exposures to tobramycin caused increases in the tobramycin MIC and an observable decrease in growth rate, but concomitant delivery of tobramycin with COS/NO at 25% of its MIC resulted in a slowing or prevention of tobramycin-resistance. The applicants likewise saw no change in the MIC of COS/NO in P. aeruginosa or MRSA after 70 or 30 passages, respectively (Table 6). Tolerance to NO may be afforded by a thicker cell membrane or wall, as Gram-positive bacteria required higher doses of NO for eradication compared to Gram-negative, but no resistance to NO was developed by continuous exposure to sub-lethal concentrations. Thus, not only can NO improve the bactericidal efficacy of antibiotics, their combined use may slow the development of antibiotic resistance.

TABLE 6

Minimum inhibitory concentrations of NO and tobramycin as single agents and of tobramycin when delivered in combination with 25% COS/NO MIC after n passages ^{a}

| Strain | Test Agent | MIC₀ (µg/mL) | MICₙ (µg/mL) | n |
|---|---|---|---|---|
| PAK | NO^{b} | 96 | 96 | 70 |
| | Tobramycin | 2 | 16 | 10 |
| | Tobramycin+NO ^{c} | 0.25 | 0.25 | 10 |
| ATCC MRSA | NO ^{b} | 384 | 384 | 30 |
| | Tobramycin | 2000 | 16000 | 10 |
| | Tobramycin+NO ^{c} | 2000 | 4000 | 10 |

^{a} single passage is defined as the exposure to serial dilutions of test agent for 24 h and subsequent dilution to 10⁶ CFU/mL. ^{b} Dose of NO from COS/NO determined by chemiluminescence. ^{c} MIC of tobramycin when delivered with 25% COS/NO MIC simultaneously.

Nitric oxide improves antibiotic action in both planktonic and biofilm bacteria and this phenomenon occurs irrespective of bacteria species, antibiotic mechanism of action, or molecular mechanisms of resistance. No antimicrobial on the market today combines broad-spectrum antimicrobial efficacy, biofilm killing, synergism with conventional antibiotics, and reversal of antibiotic resistance in an “all-in-one” therapy. In a world where antibiotic stewardship must be tightly controlled and multidrug-resistant species abound, these data suggest an enormous change in the future of antibiotic treatment. Future studies will investigate the effects of antibiotic-NO in complex infection models, such as the ex vivo porcine lung model [Harrison, F., Muruli, A., Higgins, S. & Diggle, S. P. Development of an ex vivo porcine lung model for studying growth Virulence, And signaling of pseudomonas aeruginosa. Infect. Immun. 82, 3312-3323 (2014)]. The incorporation of NO-based therapies in addressing resistant infections may allow for the use of previously ineffective antibiotic treatments, improve clinical outcomes and slow the spread of resistance.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method of increasing susceptibility of a microorganism to at least one antibiotic, comprising:
contacting the microorganism with a nitric oxide-releasing chitosan oligosaccharide (COS/NO) and
the at least one antibiotic.

2. The method of claim 1, wherein the contacting is sequential, wherein sequentially contacting comprises contacting the nitric oxide-releasing chitosan oligosaccharide (COS/NO) for a period, and subsequently contacting the organism with the at least one antibiotic.

3. The method of claim 1 or 2, wherein the microorganism is selected from the genera consisting of: Enterococcus, Staphylococcus, Klebsiella, Acinetobacter, Pseudomonas, and Enterobacter.

4. The method of any of claims 1–3, wherein the microorganism is selected from the group consisting of: Enterococcus faecium, Staphylococcus aureus, Klebsiella pneumoniae, Acinetobacter baumannii, Pseudomonas aeruginosa, and Enterobacter spp.

5. The method of any one of claims 1–4, wherein the COS/NO:
has a minimum inhibitory concentration (MIC) relative to the microorganism; and
is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

6. The method of any one of claims 1-5, wherein the at least one antibiotic:
has a minimum inhibitory concentration (MIC) relative to the microorganism; and
is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

7. The method of any one of claims 2–6, wherein the period is selected from the group consisting of at least 0.25, at least 0.5, at least 0.75, at least 1, at least 1.25, at least 1.5, at least 1.75, at least 2, at least 2.25, at least 2.5, at least 2.75, at least 3, at least 3.25, at least 3.5, at least 3.75, at least 4, at least 4.25, at least 4.5, at least 4.75, at least 5, at least 5.25, at least 5.5, at least 5.75, at least 6, at least 6.25, at least 6.5, at least 6.75, at least 7, at least 7.25, at least 7.5, at least 7.75, and at least 8 hours.

8. The method of any one of claims 1–7, wherein increased susceptibility is lower viability of the microorganism, measured as colony-forming units (CFU) per unit volume, after said contacting with the COS/NO for a period and subsequent contacting with the at least one antibiotic, as compared to contacting with either the COS/NO alone or the at least one antibiotic alone.

9. The method of any one of claims 1–8, wherein the microorganism is in a biofilm.

10. The method of any one of claims 1–9, wherein the COS/NO is in a composition formulated for: topical, oral, nasal, ophthalmic, intrathecal, parenteral, intraperitoneal, intravenous, subcutaneous, or intramuscular administration.

11. The method of any one of claims 1–10, wherein the at least one antibiotic is in a composition formulated for: topical, oral, nasal, ophthalmic, intrathecal, parenteral, intraperitoneal, intravenous, subcutaneous, or intramuscular administration.

12. The method of any one of claims 10–11, wherein the formulation is in the form of a paste, a liquid, a cream, a gel, a salve, a foam, an aerosol, a lotion, an ointment, a soap, a shampoo, a surgical drape, a suture, a bandage, a gauze, or a medical implant.

13. The method of any one of claims 1–12, wherein the at least one antibiotic is selected from the group consisting of: aminoglycosides, ansamycins, beta-lactams, carbacephems, carbapenems, cephalosporins, fluoroquinolones, glycopeptides, lincosamides, macrolides, monobactams, oxazolidinones, penicillins, phenicols, polypeptides, quinolones, streptogramins, sulfonamides, and tetracyclines.

14. The method of any one of claims 1–13, wherein the at least one antibiotic is selected from the group consisting of: aztreonam, ceftazidime, ciprofloxacin, colistin, meropenem, and tobramycin.

15. A method of reducing the development or progression, in a microorganism, of resistance to at least one antibiotic, comprising:
contacting the microorganism with a nitric oxide-releasing chitosan oligosaccharide (COS/NO) and the at least one antibiotic.

16. The method of claim 15, wherein the contacting the microorganism with a nitric oxide-releasing chitosan oligosaccharide (COS/NO) and the at least one antibiotic provides a synergistic microbicidal effect.

17. The method of claim 15 or 16, wherein contacting the microorganism with the nitric oxide-releasing chitosan oligosaccharide (COS/NO) and the at least one antibiotic occurs concurrently, sequentially, or any combination thereof.

18. The method of any of claim 17, wherein sequentially contacting comprises contacting the nitric oxide-releasing chitosan oligosaccharide (COS/NO) for a period, and subsequently contacting the organism with at least one antibiotic.

19. The method of any of claims 15–18, wherein the microorganism is selected from the genera consisting of: Enterococcus, Staphylococcus, Klebsiella, Acinetobacter, Pseudomonas, and Enterobacter.

20. The method of any of claims 15–19, wherein the microorganism is selected from the group consisting of: Enterococcus faecium, Staphylococcus aureus, Klebsiella pneumoniae, Acinetobacter baumannii, Pseudomonas aeruginosa, and Enterobacter spp.

21. The method of any one of claims 15–20, wherein the COS/NO:
has a minimum inhibitory concentration (MIC) relative to the microorganism; and
is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

22. The method of any one of claims 15–21, wherein the at least one antibiotic:
has a minimum inhibitory concentration (MIC) relative to the microorganism; and
is provided in an amount selected from about 0.1 to about 4, about 0.2 to about 4, about 0.25 to about 4, about 0.3 to about 4, about 0.4 to about 4, about 0.5 to about 4, about 0.6 to about 4, about 0.7 to about 4, about 0.75 to about 4, about 0.8 to about 4, about 0.9 to about 4, about 1 to about 4, about 2 to about 4, about 3 to about 4, about 0.1 to about 3, about 0.1 to about 2, about 0.1 to about 1, about 0.1 to about 0.9, about 0.1 to about 0.8, about 0.1 to about 0.75, about 0.1 to about 0.7, about 0.1 to about 0.6, about 0.1 to about 0.5, about 0.1 to about 0.4, about 0.1 to about 0.3, about 0.1 to about 0.25, about 0.1 to about 0.2, about 0.1, about 0.2, about 0.25, about 0.3, about 0.4, about 0.5, about 0.6, about 0.7, about 0.75, about 0.8, about 0.9, about 1, about 2, about 3, and about 4 MIC.

23. The method of any one of claims 18–22, wherein the period is selected from the group consisting of at least 0.25, at least 0.5, at least 0.75, at least 1, at least 1.25, at least 1.5, at least 1.75, at least 2, at least 2.25, at least 2.5, at least 2.75, at least 3, at least 3.25, at least 3.5, at least 3.75, at least 4, at least 4.25, at least 4.5, at least 4.75, at least 5, at least 5.25, at least 5.5, at least 5.75, at least 6, at least 6.25, at least 6.5, at least 6.75, at least 7, at least 7.25, at least 7.5, at least 7.75, and at least 8 hours.

24. The method of any one of claims 15–23, wherein the reducing the development or progression comprises increased susceptibility of the microorganism to the at least one antibiotic by lowering viability of the microorganism, measured as colony-forming units (CFU) per unit volume, after said contacting with the COS/NO for a period and subsequent contacting with the at least one antibiotic, as compared to contacting with either the COS/NO alone or the at least one antibiotic alone.

25. The method of any one of claims 15–24, wherein the microorganism is in a biofilm.

26. The method of any one of claims 15-25, wherein the COS/NO is in a composition formulated for: topical, oral, nasal, ophthalmic, intrathecal, parenteral, intraperitoneal, intravenous, subcutaneous, or intramuscular administration.

27. The method of any one of claims 15–26, wherein the at least one antibiotic is in a composition formulated for: topical, oral, nasal, ophthalmic, intrathecal, parenteral, intraperitoneal, intravenous, subcutaneous, or intramuscular administration.

28. The method of any one of claims 26–27, wherein the formulation is in the form of a paste, a liquid, a cream, a gel, a salve, a foam, an aerosol, a lotion, an ointment, a soap, a shampoo, a surgical drape, a suture, a bandage, a gauze, or a medical implant.

29. The method of any one of claims 15–28, wherein the at least one antibiotic is selected from the group consisting of: aminoglycosides, ansamycins, beta-lactams, carbacephems, carbapenems, cephalosporins, fluoroquinolones, glycopeptides, lincosamides, macrolides, monobactams, oxazolidinones, penicillins, phenicols, polypeptides, quinolones, streptogramins, sulfonamides, and tetracyclines.

30. The method of any one of claims 15–29, wherein the at least one antibiotic is selected from the group consisting of: aztreonam, ceftazidime, ciprofloxacin, colistin, meropenem, and tobramycin.

31. The method of any one of claims 1–30, wherein said (COS/NO) comprises at least one structural unit of formula (I), 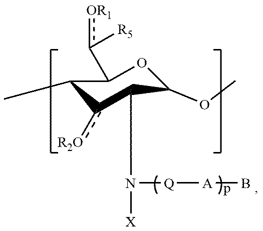
and optionally, at least one structural unit of formula (II), 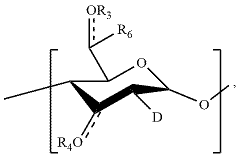
wherein,
R₁, R₂, R₃ and R₄, if present, are each independently selected from the group consisting of hydrogen; C₁₋₅ alkyl(C═O)—, when the C₁₋₅ alkyl is methyl, Me(C═O)— is an acyl, Ac; and C₁₋₅ alkyl; 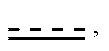
in each instance, is a single or double bond,
wherein in each instance where 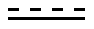
is a double bond, R
₁, R₂, R₃ or R₄ attached to the double bond-O is absent;
when R₁ is absent, R₅ is hydrogen, hydroxyl, C₁₋₅ alkyl or C₁₋₅ alkoxy;
when R₃ is absent, R₆ is hydrogen, hydroxyl, C₁₋₅ alkyl or C₁₋₅ alkoxy;
wherein in each instance where 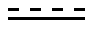
is a single bond, R
₁, R₂, R₃ or R₄ attached to the double bond-O is present;
when R₁ is present, R₅ is hydrogen;
when R₃ is present, R₆ is hydrogen;
Q is —(CR_{c}R_{d})ᵥ—;
wherein R_{c} and R_{d} are independently hydrogen or C₁₋₅ alkyl; and v is an integer from 2 to 6;
p is an integer from 1 to 100;
A is 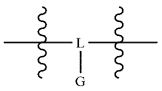
wherein, L is S, O or N; and G, in each instance, is independently, hydrogen, or is taken together with L to form a nitric oxide donor;
X is hydrogen, C₁₋₅ alkyl or is taken together with N to form a nitric oxide donor;
B is hydrogen or —Y—Z, wherein Y is a spacer and Z is a polymer or a terminus group; or B is absent;
D is —NRₐR_{b}, wherein Rₐ and R_{b} are independently selected from the group consisting of hydrogen, formyl, C₁₋₅ alkyl(C═O)—, when the C₁₋₅ alkyl is methyl, Me(C═O)— is an acyl, Ac, C₁₋₅ alkyl and C₁₋₅ alkyl ester;
or D is 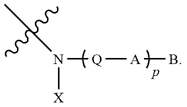
.

32. The method of claim 31, wherein
R₁, R₂, R₃, R₄, R₅, and R₆, are each hydrogen; 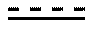
is a single bond;
Q is —(CR_{c}R_{d})ᵥ—; wherein R_{c} and R_{d} are hydrogen and v is 2;
p is an integer from 1 to 10;
A is 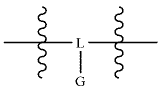
wherein, L is N and G is hydrogen;
X is taken together with N to form a nitric oxide donor;
B is hydrogen; and
D is —NRₐR_{b}, wherein Rₐ and R_{b} are each hydrogen.

33. The method of claim 32, wherein p is 1.

34. The method of any one of claims 31–33, wherein the nitric oxide donor taken together with the atom on the COS/NO to which it is bound is selected from the group consisting of a diazeniumdiolate, nitrosothiol, a nitrosamine, a hydroxyl nitrosamine, a hydroxyl amine, a hydroxyurea, and combination thereof.

35. The method of any one of claims 31–34, wherein the nitric oxide donor is diazeniumdiolate.

36. The method of any one of claims 31–35, wherein the COS/NO has a total releasable nitric oxide storage of at least 0.5 µmol of NO per milligram of the COS/NO as determined in aqueous buffer at pH 7.4 and 37° C.

37. The method of any one of claims 31–36, wherein the COS/NO has a total releasable nitric oxide storage in a range of about 0.5 µmol to 2.5 µmol of NO per milligram of the COS/NO as determined in aqueous buffer at pH 7.4 and 37° C.

38. The method of any one of claims 31–37, wherein the COS/NO has a half-life for nitric oxide release in a range of between about 0.7-4.2 hours as determined in aqueous buffer at pH 7.4 and 37° C.

39. The method of any one of claims 31–38, wherein the COS/NO has a half-life for nitric oxide release over about 1 hour as determined in aqueous buffer at pH 7.4 and 37° C.

40. The method of any one of claims 31–39, wherein the COS/NO has a total NO release after 4 hours in a range of between about 0.3-2.0 µmol of NO per milligram of the COS/NO as determined in aqueous buffer at pH 7.4 and 37° C.
